(19) United States
(12) Patent Application Publication
(10) Pub. No.: US 2022/0082523 A1
(43) Pub. Date: Mar. 17, 2022

(54) GAS SENSOR AND METHOD FOR MANUFACTURING THE SAME

(71) Applicant: NATIONAL SUN YAT-SEN UNIVERSITY, Kaohsiung City (TW)

(72) Inventors: Ting-Chang Chang, Kaohsiung City (TW); Yi-Ting Tseng, Kaohsiung City (TW); Chun-Chu Lin, Kaohsiung City (TW); Wen-Chung Chen, Kaohsiung City (TW); Po-Hsun Chen, Kaohsiung City (TW); Shih-Kai Lin, Kaohsiung City (TW)

(21) Appl. No.: 17/088,754

(22) Filed: Nov. 4, 2020

(30) Foreign Application Priority Data
Sep. 11, 2020 (TW) 109131323

(51) Int. Cl. G01N 27/414; G01N 33/00

(52) U.S. Cl. CPC G01N 27/4141; G01N 33/0036; G01N 33/0031

(57) ABSTRACT

A gas sensor and a method for manufacturing the gas sensor are disclosed. The gas sensor includes a substrate, a heating member disposed on the substrate, a sensing layer covering the heating member, and two electrodes respectively electrically connected to the sensing layer. The sensing layer includes a doping element with an electronegativity greater than 2. The method for manufacturing the gas sensor includes a deposition process stacking a heating member, a sensing layer, and two electrodes on a substrate by deposition, and a doping process introducing a doping gas when depositing the sensing layer.

### CROSS REFERENCE TO RELATED APPLICATION

The application claims the benefit of Taiwan application serial No. 109131323, filed on Sep. 11, 2020, and the entire contents of which are incorporated herein by reference.

### BACKGROUND OF THE INVENTION

### 1. Field of the Invention

The present invention relates to an environmental monitoring technology and, more particularly, to a gas sensor and a manufacturing method thereof for improving sensing capability and reducing environmental humidity interference.

### 2. Description of the Related Art

Gas sensing technology is commonly used for things that are closely related to life, such as monitoring air quality, detecting the leakage of dangerous industrial gases, and alcohol concentration testing to prevent drunk driving. The technology of the operation mechanism of gas sensing can be roughly divided into technical areas of semiconductor, infrared light and electrochemistry. Among them, semiconductor chip gas sensors can be produced through micro electro mechanical system (MEMS) process, which can achieve the advantages of miniaturization, low energy consumption and mass production. Through the printing process, soft and flexible electrochemical gas sensors can be produced. The above-mentioned different sensing technologies can be respectively applied to detect gases of different compositions and be installed in various devices.

The above-mentioned conventional gas sensors, such as an electrochemical breath alcohol tester uses a sensing component made of gas-sensitive material to react with the gas to be measured to generate an electric current signal related to gas concentration. However, after the conventional gas sensor is used for a long time, the sensitivity of the sensing component will be greatly attenuated, which will cause the measurement result to lose stability and accuracy, and increase the probability of sensing failure and reading error. In addition, the conventional gas sensor is more susceptible to environmental humidity interference after aging, thus reducing the sensing capability of the target gas.

In light of this, it is necessary to improve the conventional gas sensor and its manufacturing method.

### SUMMARY OF THE INVENTION

To solve the above disadvantages, it is an objective of the present invention to provide a gas sensor, which can increase the sensing sensitivity of the target gas.

It is another objective of the present invention to provide a gas sensor, which can reduce the influence of environmental humidity on gas sensing.

It is a further objective of the present invention to provide a method for manufacturing a gas sensor, which can restore the sensitivity of the gas sensor and prolong the service life.

It is yet another objective of the present invention to provide a method for manufacturing a gas sensor, which can simplify the process and reduce the cost.

As used herein, the term “a” or “an” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

A gas sensor according to the present invention includes a substrate, a heating member disposed on the substrate, a sensing layer covering the heating member, and two electrodes respectively electrically connected to the sensing layer. The sensing layer includes a doping element with an electronegativity greater than 2.

A method for manufacturing the gas sensor according to the present invention includes a deposition process stacking a heating member, a sensing layer, and two electrodes on a substrate by deposition, and a doping process introducing a doping gas when depositing the sensing layer. The doping gas includes a doping element with an electronegativity greater than 2.

Accordingly, the gas sensor and the method for manufacturing the gas sensor of the present invention can improve the sensitivity of the reaction between the sensing layer and the gas to be measured by doping elements with high electronegativity into the sensing layer. In addition, through doping and modification, the gas sensor can be restored to its original measurement stability and accuracy after a long time of use, and the influence of environmental humidity on gas sensing sensitivity can be reduced, having the effects of improving the sensing capability and prolonging the service life of the gas sensor.

In an example, the heating member is disposed on a first surface of the substrate, and the substrate has a cavity located on a second surface opposite to the first surface of the substrate. Thus, the cavity can be used as a thermal insulation space, having the effect of preventing heat from being transmitted out of the gas sensor.

In an example, the heating member is electrically connected to a power source. The heating member converts an electric energy of the power source into a heat and transmits the heat to the sensing layer. Thus, the temperature of the sensing layer can be raised by the heating member, having the effect of providing a high temperature state required for gas sensing.

In an example, the doping element is fluorine, chlorine, sulfur or oxygen. Thus, the doping element has large electronegativity and is easy to obtain, having the effects of simplifying the process and reducing the cost.

In an example, an atomic percentage of the doping element in the sensing layer is 1%˜10%. Thus, the sensing layer can maintain surface characteristics and increase the capability to attract electrons, having the effect of improving the sensing sensitivity.

In an example, each of the two electrodes is electrically isolated from the heating member. Thus, the power source of the heating member can be prevented from interfering with the electric current value measured by the two electrodes, having the effect of improving the measurement accuracy.

In an example, the gas sensor according to the present invention further includes an insulating film disposed between the substrate and the heating member. Thus, the insulating film can withstand high temperatures and reduce electrical interference, having the effects of maintaining safety in use and reducing the probability of misjudgment.

In an example, the doping gas is oxygen, chlorine, fluorocarbon, ammonia, hydrogen sulfide, hydrogen selenide, hydrogen chloride, hydrogen bromide, hydrogen iodide, ammonium chloride, carbamide, or a combination of gases selected therefrom. Thus, the doping gas can be supplied at room temperature and is suitable for the original deposition process conditions, having the effects of simplifying the process and reducing the cost.

In an example, the doping process is used to modify a manufactured gas sensor and repair a worn out gas sensor which has been used for a long time. Thus, the sensing layer can be repeatedly processed through the doping process, having the effects of restoring the sensing sensitivity and prolonging the service life.

### BRIEF DESCRIPTION OF THE DRAWINGS

The present invention will become clearer in light of the following detailed description of illustrative embodiments of this invention described in connection with the drawings.

FIG. 1 is an exploded perspective view of a preferred embodiment of the present invention.

FIG. 2 is a comparison diagram of sensing results of a preferred embodiment of the present invention and of the prior art sensing alcohol concentration.

FIG. 3 is a comparison diagram of sensing results of a preferred embodiment of the present invention and of the prior art when affected by humidity.

### DETAILED DESCRIPTION OF THE INVENTION

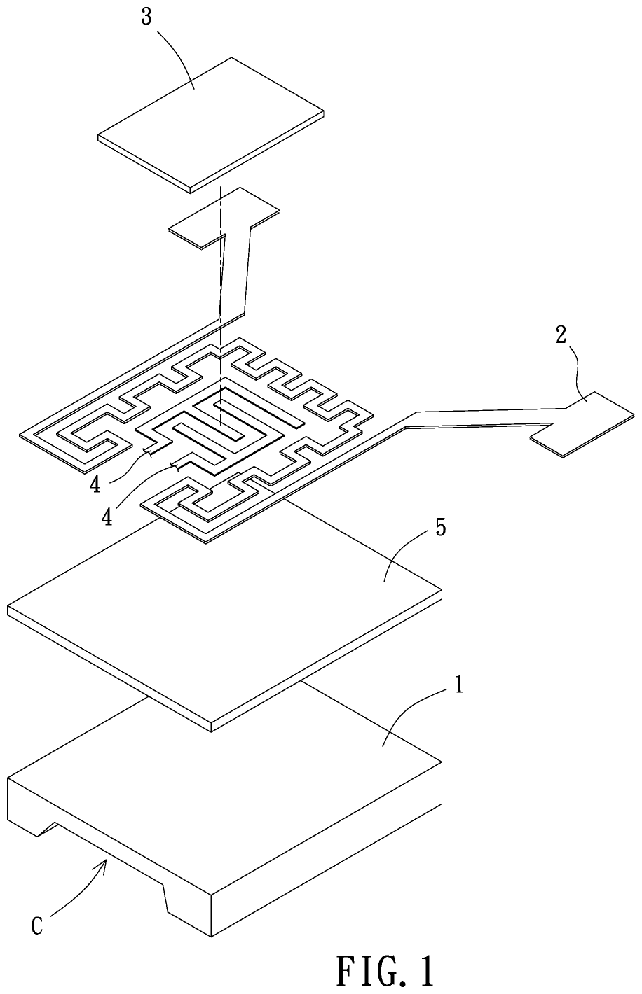

Please refer to FIG. 1, which is a preferred embodiment of a gas sensor of the present invention, which includes a substrate 1, a heating member 2, a sensing layer 3 and two electrodes 4. The heating member 2 is disposed on the substrate 1, the sensing layer 3 covers the heating member 2, and the two electrodes 4 are respectively electrically connected to the sensing layer 3.

The substrate 1 is used to carry various electronic components and circuits, which can be formed on the substrate 1 with materials such as metals, semiconductors and insulators by semiconductor process technologies such as sputtering, evaporation deposition, chemical vapor deposition, and ion implantation. The heating member 2 is disposed on a first surface of the substrate 1. The substrate 1 may have a cavity C as a thermal insulation space and the cavity C is preferably located on a second surface opposite to the first surface of the substrate 1, so that the heat generated by the heating member 2 can be dissipated to the cavity C, and the heat can also be prevented from being transmitted out of the gas sensor. The substrate 1 can be a silicon substrate with good thermal conductivity and thermal stability, which can avoid malfunction of the electronic components even in a high temperature working environment.

The heating member 2 is electrically connected to a power source (not shown), which can convert electric energy of the power source into heat to provide a high temperature state required for gas sensing. The heating member 2 can be wound by metal materials to increase the contact area between the heating member 2 and other components, so as to improve the efficiency of heat transfer.

The main material of the sensing layer 3 is metal oxide semiconductor (MOS) materials, such as tin dioxide (SnO₂), zinc oxide (ZnO), nickel oxide (NiO), iron oxide (Fe₂O₃), etc. Different materials can be selected for the sensing layer 3 according to different gases to be measured. The sensing layer 3 includes a doping element with an electronegativity (EN) greater than 2. The doping element can be fluorine (F), chlorine (Cl), sulfur (S), oxygen (O), etc., which has strong attraction to electrons, so as to improve the capability of capturing ionization electrons on the surface of the sensing layer 3. The atomic percentage (Atom %) of the doping element doped in the sensing layer 3 is preferably 1%˜10%. When the sensing layer 3 is at a working temperature of 200° C.˜400° C., the resistance value of the sensing layer 3 is changed by the oxidation or reduction reaction between the surface of the metal oxide semiconductor of the sensing layer 3 and the gas to be measured.

The two electrodes 4 are used to measure the electric current passing through the surface of the sensing layer 3, which can record the change of the resistance value of the sensing layer 3. The two electrodes 4 can also be cross-arranged fork-shaped electrodes, which can simultaneously collect electric current values at different positions on the surface of the sensing layer 3, having the effect of improving measurement sensitivity and accuracy. Further, each of the two electrodes 4 is preferably electrically isolated from the heating member 2 to prevent the power source of the heating member 2 from interfering with the electric current value measured by the two electrodes 4.

The gas sensor of the present invention can also have an insulating film 5, which is disposed between the substrate 1 and the heating member 2. The material of the insulating film 5 can be silicon dioxide (SiO₂) with high temperature resistance and electrical insulation characteristics, so that the insulating film 5 can withstand high temperatures during sensing and reduce the influence of electrical interference on the measurement.

The gas sensor of the present invention can raise the temperature of the sensing layer 3 with the heating member 2, and set different working temperatures corresponding to different gases to be measured. Then, the sensing layer 3 contacts the gas to be measured and reacts to form ionized electrons, which increases the electric conductivity on the surface of the sensing layer 3. And then, by measuring the electric current passing through the two electrodes 4, the resistance value on the surface of the sensing layer 3 and the concentration of the corresponding gas to be measured can be obtained.

A preferred embodiment of the manufacturing method of the gas sensor of the present invention includes a deposition process in which the heating member 2, the sensing layer 3, the two electrodes 4 and the insulating film 5 are stacked on the substrate 1 by a deposition method. The manufacturing method also includes a doping process in which a doping gas is introduced when the sensing layer 3 is deposited. The doping gas includes the doping element, so as to make an atomic percentage of the doping element in the sensing layer 3 at around 1%˜10%.

When depositing the heating member 2, the two electrodes 4 and the insulating film 5, argon (Ar) gas can be introduced and the target material corresponding to each layer can be used. For example, platinum (Pt) can be used for the heating member 2 and conducting materials such as copper or silver can be used for the two electrodes 4, but the present invention is not limited hereto.

When depositing the sensing layer 3, argon gas and the doping gas are introduced at the same time, and corresponding materials, such as tin dioxide, zinc oxide, nickel oxide, iron oxide, etc., are used as target materials according to different gases to be measured. The working pressure of the argon gas can be 8 mTorr, the working pressure of the doping gas can be 4 mTorr, and the doping gas can be oxygen (O₂), chlorine (Cl₂), fluorocarbon (CₙFₙ, n is an integer greater than 0), ammonia (NH₃), hydrogen sulfide (H₂S), hydrogen selenide (H₂Se), hydrogen chloride (HCl), hydrogen bromide (HBr), hydrogen iodide (HI), ammonium chloride (NH₄Cl), carbamide (CO(NH₂)₂, urea) or a combination of gases selected from the above, which includes elements such as oxygen, chlorine, fluorine, sulfur respectively.

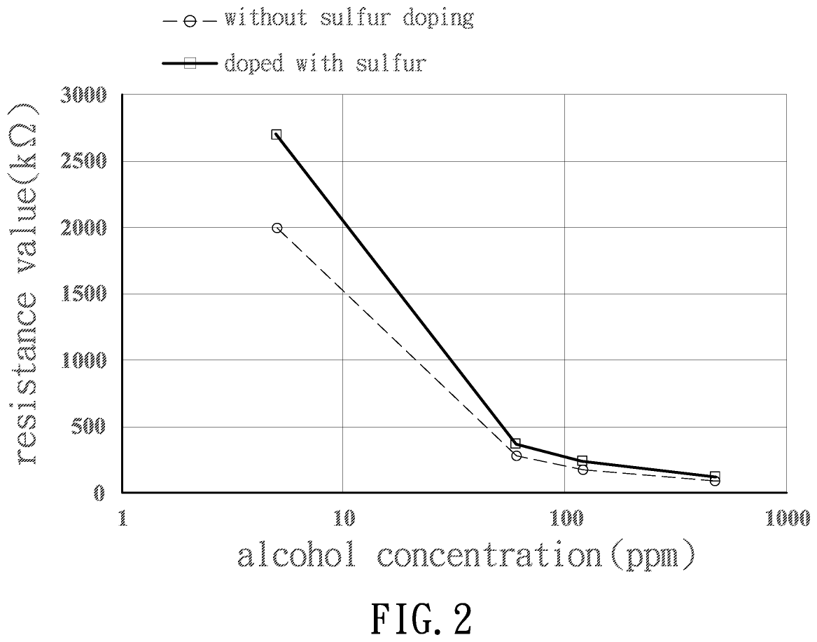

Please refer to FIG. 2, which is a comparison of sensing results of gas sensors without and with sulfur doping in different alcohol concentrations. The results show that the resistance values measured by the gas sensor doped with sulfur are relatively high, while the change rate of resistance values of the gas sensor doped with sulfur is greatly increased, especially when the alcohol concentration is less than 10 ppm, thus increasing the sensitivity of alcohol sensing.

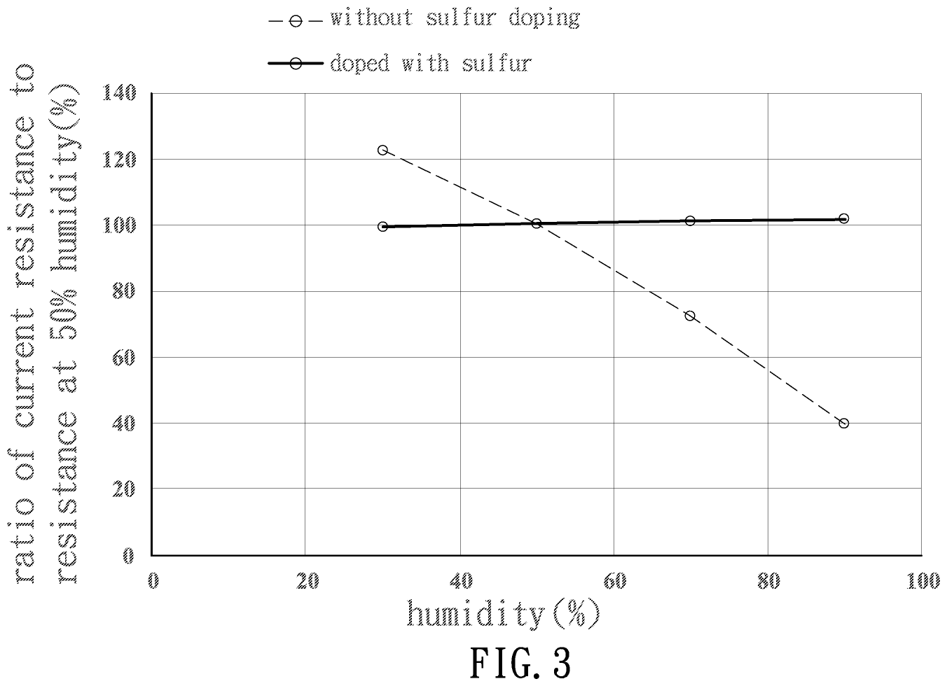

Please refer to FIG. 3, which is a comparison of sensing results of gas sensors without and with sulfur doping in different humidity environments. The results show that the resistance values measured by the gas sensor without sulfur doping decrease as the humidity increases, while the resistance values measured by the gas sensor doped with sulfur have a relatively small change corresponding to the humidity change, thus reducing the influence of environmental humidity on the sensitivity of the gas sensor.

The doping process can not only complete doping during the process of depositing the sensing layer 3, but also modify the manufactured gas sensor and repair a worn out gas sensor which has been used for a long time, thus having the effects of restoring the sensing sensitivity and prolonging the service life.

In summary, the gas sensor and manufacturing method of the gas sensor of the present invention can improve the sensitivity of the reaction between the sensing layer and the gas to be measured by doping elements with high electronegativity into the sensing layer. In addition, through doping and modification, the gas sensor can be restored to its original measurement stability and accuracy after a long time of use, and the influence of environmental humidity on gas sensing sensitivity can be reduced, thus having the effects of improving the sensing capability and prolonging the service life of the gas sensor.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. A gas sensor, comprising:
a substrate;
a heating member disposed on the substrate;
a sensing layer covering the heating member, wherein the sensing layer includes a doping element with an electronegativity greater than 2; and
two electrodes respectively electrically connected to the sensing layer.

2. The gas sensor as claimed in claim 1, wherein the heating member is disposed on a first surface of the substrate, and the substrate has a cavity located on a second surface opposite to the first surface of the substrate.

3. The gas sensor as claimed in claim 1, wherein the heating member is electrically connected to a power source, and wherein the heating member converts an electric energy of the power source into a heat and transmits the heat to the sensing layer.

4. The gas sensor as claimed in claim 1, wherein the doping element is fluorine, chlorine, sulfur or oxygen.

5. The gas sensor as claimed in claim 1, wherein an atomic percentage of the doping element in the sensing layer is 1%˜10%.

6. The gas sensor as claimed in claim 1, wherein each of the two electrodes is electrically isolated from the heating member.

7. The gas sensor as claimed in claim 1, further comprising an insulating film disposed between the substrate and the heating member.

8. A method for manufacturing a gas sensor, comprising:
a deposition process stacking a heating member, a sensing layer, and two electrodes on a substrate by deposition; and
a doping process introducing a doping gas when depositing the sensing layer, wherein the doping gas includes a doping element with an electronegativity greater than 2.

9. The method for manufacturing a gas sensor as claimed in claim 8, wherein the doping gas is oxygen, chlorine, fluorocarbon, ammonia, hydrogen sulfide, hydrogen selenide, hydrogen chloride, hydrogen bromide, hydrogen iodide, ammonium chloride, carbamide, or a combination of gases selected therefrom.

10. The method for manufacturing a gas sensor as claimed in claim 8, wherein the doping process is used to modify a manufactured gas sensor and repair a worn out gas sensor which has been used for a long time.
